(19) United States
(12) Patent Application Publication
(10) Pub. No.: US 2010/0075933 A1
(43) Pub. Date: Mar. 25, 2010

(54) INJECTABLE COMPOSITIONS OF VITAMIN D COMPOUNDS

(76) Inventors: Sunita Vijay Shelke, Aurangabad (IN); Subhasish Panda, Paschim Medinipur (IN); Bikash Kumar Sahoo, Cuttack (IN); Ravikumar Namballa, Hyderabad (IN); Hiren Patel, Ahmedabad (IN); Suryakumar Jayanthi, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/509,697

(22) Filed: Jul. 27, 2009

Related U.S. Application Data
(60) Provisional application No. 61/102,077, filed on Oct. 2, 2008.

(30) Foreign Application Priority Data
Jul. 28, 2008 (IN) 1700/CHE/2008

(51) Int. Cl. A61K 31/59

(52) U.S. Cl. 514/167

(57) ABSTRACT

Pharmaceutical injectable compositions comprising at least one vitamin D compound as an active agent, processes for preparing such compositions, and methods of using such compositions.

Aspects of the present invention relate to pharmaceutical injectable compositions comprising at least one vitamin D compound as an active agent, and processes for preparing such compositions. Further aspects relate to methods of using such compositions.

Several pharmacologically active vitamin D compounds are hydrophobic or lipophilic and are only sparingly or negligibly water-soluble. The poor water solubility of these active agents often results in major difficulties in formulation particularly when easily sterilizable and administrable homogeneous aqueous solutions are needed. Also the poor water solubility of these active agents raises concerns of stability upon storage and especially throughout the shelf-life of the product.

The lipophilicity of the vitamin D compounds makes it difficult to manufacture an efficacious formulation, particularly, an injectable formulation, which is preferred, for example, in renal dialysis patients.

Additionally, vitamin D compounds are known to be oxygen-sensitive. They get oxidized when exposed to air, and thus, loose integrity. One approach to circumvent this problem is to add an antioxidant directly to a formulation of the drug. However, certain antioxidants, such as ascorbic acid and sodium ascorbate, which are highly water soluble, will discolor in the course of performing their intended function. Buffers and/or chelating agents have also been added to decrease oxygen sensitivity thus maintaining active drug potency (e.g., U.S. Pat. Nos. 4,308,264, 4,948,788, and 5,182,274). However, the buffers and chelating agents are known to introduce undesirable levels of aluminum into the product. Thus, there is a need for pharmaceutical injectable formulations of vitamin D compounds that overcome the limitations of the prior art compositions.

Paricalcitol is a synthetically manufactured analog of calcitriol, the metabolically active form of vitamin D, and is indicated for the prevention and treatment of secondary hyperparathyroidism in chronic kidney disease. It has chemical names 19-nor-1α,3β,25-trihydroxy-9,10-secoergosta-5(Z),7(E),22(E)-triene, and (1R,3R,7E,17β)-17-[(1R,2E,4S)-5-hydroxy-1,4,5-trimethylhex-2-en-1-yl]-9,10-secoestra-5,7-diene-1,3-diol, and has structural Formula I.

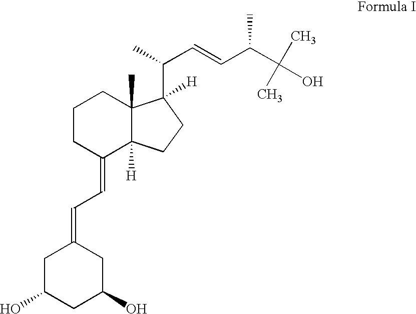

A commercially available injectable paricalcitol product is sold as ZEMPLAR®, by Abbott. The ZEMPLAR® product is available as a sterile, clear and colorless aqueous solution for intravenous injection, indicated for the prevention and treatment of secondary hyperparathyroidism in chronic kidney disease.

H. Whittle et al., “Intravenous vitamin D in the detection of vitamin-D deficiency,” The Lancet, pages 747-750, Apr. 12, 1969, discloses an injectable composition of Vitamin D3, anhydrous ethanol and propylene glycol.

Bertino et al., “Stability of an extemporaneous formulation of injectable cholecalciferol”, American Journal of Hospital Pharmacy, Volume 38(12), pages 1932-1933, 1981, discloses an injection composition comprising vitamin D derivative, propylene glycol and ethanol, stored in glass vials.

U.S. Pat. No. 5,587,497 discloses vitamin D-related compounds, namely the 1α-hydroxy-19-nor-vitamin D analogs including paricalcitol, as well as a general method for their chemical synthesis.

U.S. Pat. No. 6,361,758 discloses a self-preserved, synergistic antimicrobial vehicle composition consisting essentially of water, a low molecular weight alcohol in the range of about 25% to about 30%, and a glycol derivative in the range of about 20% to about 25%.

U.S. Pat. No. 6,136,799 discloses an aqueous pharmaceutical self-preserved composition for parenteral administration having paricalcitol, about 50% (v/v) of an organic solvent and about 50% (v/v) water.

U.S. Patent Application Publication No. 2007/0166187 discloses a method of preventing the decomposition of a pharmaceutical composition by storing in a glass vial sealed with a stopper comprising a halogenated butyl polymer selected from the group comprising of chlorobutyl, chlorinated butyl, fluorobutyl, fluorinated butyl and mixtures thereof.

U.S. Patent Application Publication Nos. 2004/0053895 and 2004/0058895 disclose a multi-use vessel comprising a pharmaceutical formulation comprising an active vitamin D compound, a non-ionic solubilizer, a lipophilic antioxidant and an aqueous vehicle.

U.S. Patent Application Publication No. 2007/0104780 discloses a pharmaceutical composition of drug with non-aqueous carrier comprising at least one phospholipid.

U.S. Patent Application Publication No. 2006/0183722 discloses a formulation comprising a vitamin D compound, a non-ionic solubilizer and a lipophilic antioxidant.

The clear criterion for the self-preserved commercial paricalcitol injectable formulation (ZEMPLAR®) is the presence of a solvent system of about 50% (v/v) water and an organic solvent selected from aliphatic alcohols of from 1 to 5 carbons in the range of from 15% to 30% (v/v) and glycol derivatives in the range of from 20% to 35% (v/v), selected from glycerin, propylene glycol and liquid polymers of glycol. Therefore, the solvent system which acts as a solubilizer and is capable of being used as a preservative system for formulating the paricalcitol into the injection formulation works in a very specific combination only.

There remains a need for injectable compositions comprising vitamin D compounds that are clinically tolerable, effective and safe, possess appreciable stability, and are cost-effective.

### SUMMARY

In an aspect, the invention provides pharmaceutical injectable compositions comprising at least one vitamin D compound, such as paricalcitol, which are clinically tolerable, effective and safe, and stable.

In an aspect, the invention provides injectable compositions comprising a therapeutically effective amount of paricalcitol, including salts, esters, isomers, solvates, hydrates and polymorphs thereof, at least one vehicle comprising water, aqueous solvents, organic solvents, hydro-alcoholic solvents, oily substances, or mixtures thereof, and optionally one or more pharmaceutically acceptable excipients.

In an aspect, the invention provides injectable compositions comprising a therapeutically effective amount of paricalcitol, including salts, esters, isomers, solvates, hydrates and polymorphs thereof, at least one vehicle wherein the vehicle comprises more than about 50% (v/v) of organic solvent and less than about 50% (v/v) of at least one other solvent, and optionally one or more pharmaceutically acceptable excipients.

In an aspect, the invention provides injectable compositions comprising a therapeutically effective amount of paricalcitol and a vehicle comprising about 50% (v/v) or more of glycol derivative, about 10% (v/v) or less of alcohol, and about 40% (v/v) or less of water.

In an aspect, the invention provides injectable compositions comprising a therapeutically effective amount of paricalcitol and a vehicle comprising about 53% (v/v) or more of propylene glycol, about 7% (v/v) or less of dehydrated alcohol, and about 40% (v/v) or less of water for injection.

In an aspect, the invention provides substantially alcohol-free, and/or substantially surfactant-free, and/or substantially preservative-free, injectable compositions comprising vitamin D compounds.

In an aspect, the invention provides injectable compositions comprising vitamin D compounds in a single-use or multi-use container.

In an aspect, the invention provides injectable compositions comprising vitamin D compounds packaged in a glass vial, sealed with a stopper comprising a butyl polymer or halogenated butyl polymer comprising bromobutyl, ethylene-propylenediene monomers, polyisoprene, or mixtures thereof.

In an aspect, the invention provides processes for the preparation of injectable compositions comprising vitamin D compounds.

In an aspect, the invention provides method of using compositions of the present invention.

### DETAILED DESCRIPTION

The present invention provides pharmaceutical injectable compositions comprising at least one vitamin D compound. In an embodiment of the present invention, the vitamin D compound comprises paricalcitol, including salts, esters, isomers, solvates, hydrates and polymorphs thereof.

The pharmaceutical formulations of vitamin D compounds, according to an embodiment of the present invention, comprise additionally a vehicle in which the vitamin D compound is solubilized, to make a solution or a dispersion, wherein the vehicle is aqueous or oily, or combinations thereof. The formulations are highly stable in the vehicles used, and provide therapeutically useful concentrations of active ingredient.

The compositions of the present invention are intended to include any biologically active vitamin D compound, including a pro-drug, a precursor, a metabolite or an analog, in any stage of its metabolism. It is known that vitamin D compounds display a variety of biological activities, e.g., in calcium and phosphate metabolism as an antineoplastic agent and as an anti-hyperparathyroid agent. Examples of vitamin D compounds suitable for formulations of the present invention include, but are not limited to, 1α,24-dihydroxyvitamin D₂, 1α,2-dihydroxyvitamin D₄, 1α,24-dihydroxyvitamin D₂, 1α,25-dihydroxyvitamin D₃ (calcitriol), 1α hydroxyvitamin D₃ (α-calcidol) 1α,25-dihydroxyvitamin D₂, 1α,25-dihydroxyvitamin D₄, and 1α,24,25-dihydroxyvitamin D₂, seocalcitol (EB-1089), calcipotriol, 22-oxacalcitriol (maxacalcitol), fluorinated compounds such as falecalcitriol, and 19-nor compounds such as paricalcitol, including any of their salts, esters, isomers, solvates, hydrates and polymorphs. Among those compounds having a chiral center, e.g., in the side chain, such as at C₂₄, it is understood that both epimers (e.g., R- and S-) and the epimeric mixture are within the scope of the present invention.

The amount of vitamin D useful in the injectable compositions according to the present invention, although not limited, can be between about 0.1 microgram and about 1000 micrograms per unit dose, or per ml of the injectable preparation, or for the entire number of doses if the composition is intended for multiple administrations.

Further the selected dosage of the vitamin D compound useful in the present invention depends on the intended activity, the duration of activity, the severity of the condition being treated, and the condition and history of the specific subject.

The compositions of the present invention are useful for therapy or prophylaxis for the management of one or more diseases or disorders in a subject in need thereof.

In an embodiment, the present invention provides injectable compositions comprising a therapeutically effective amount of a vitamin D compound comprising paricalcitol or its esters, isomers, solvates, hydrates or polymorphs; at least one vehicle comprising aqueous solvents, hydro-alcoholic solvents, oily substances, or mixtures thereof; and optionally one or more pharmaceutically acceptable excipients.

In an embodiment, the present invention provides injectable compositions comprising a therapeutically effective amount of paricalcitol or its esters, isomers, solvates, hydrates or polymorphs, at least one vehicle wherein the vehicle comprises more than about 50% (v/v) of organic solvent and less than about 50% (v/v) of at least one other solvent, and optionally one or more pharmaceutically acceptable excipients.

In another embodiment, the present invention provides injectable compositions comprising a therapeutically effective amount of paricalcitol and a vehicle comprising about 50% (v/v) or more of glycol derivative, about 10% (v/v) or less of alcohol and about 40% (v/v) or less of water.

In another embodiment, the present invention provides injectable compositions comprising a therapeutically effective amount of paricalcitol and a vehicle comprising about 53% (v/v) or more of propylene glycol, about 7% (v/v) or less of dehydrated alcohol, and about 40% or less of water for injection.

According to an embodiment of the present invention, a glycol derivative comprises a low-molecular weight glycol such as glycerin, a polyethylene glycol (PEG), a propylene glycol (PG), and the like, or any mixtures thereof.

Suitable alcohols that are useful in the present invention include, but are not limited to, one or more of Cᵢ to C₈ aliphatic or aromatic alcohols such as dehydrated alcohol or ethanol, propanol, butanol, benzyl alcohol, and the like, and one or more of substituted Cᵢ to C₈ aliphatic or aromatic alcohols.

The pharmaceutically acceptable excipients that are useful in the present invention include, but are not limited to, preservatives, non-aqueous vehicles, chelating agents, surface-active agents (surfactants), antioxidants, reducing agents, buffers, pH adjusting agents, tonicity adjustors, and any other parenterally acceptable excipients known to the art, including any mixtures thereof.

It should be understood that the vehicle and/or the other pharmaceutically acceptable excipients of the present invention can perform one or more than one function, for example, an alcohol, if used in the composition, might also act as a preservative.

Suitable surface-active agents of the present invention include, but are not limited to, one or more of amphoteric, non-ionic, cationic or anionic surfactants. Examples of such surfactants are polysorbates, sodium lauryl sulfate, monooleate, monolaurate, monopalmitate, monostearate or another ester of polyoxyethylene sorbitane, sodium dioctylsulfosuccinate (DOSS), lecithin, stearylic alcohol, cetostearylic alcohol, cholesterol, polyoxyethylene ricin oil, polyoxyethylene fatty acid glycerides, poloxamers, polyethoxylated hydrogenated castor oils such as Cremophor™ RH 40, and the like.

Suitable preservatives include one or more of benzyl alcohol, methyl paraben, propyl paraben, benzyl paraben, and the like.

Suitable non-aqueous vehicles include one or more of polysorbates, polyethoxylated castor oil, oils like castor oil or sesame oil, benzyl alcohol, and the like.

Suitable chelating agents include one or more of inorganic or organic phosphates or citrates, ethylenediaminetetraacetic acid, diethylenetriamine-pentaacetic acid, dimercaptosuccinic acid, deferoxamine, and the like.

Suitable antioxidants and reducing agents include one or more of ascorbic acid, gentisic acid, tocopherol-derived compounds, butylated hydroxyanisole, butylated hydroxytoluene, citric acid, and the like.

Suitable buffers and pH adjusting agents include one or more of hydrochloric acid, sulfuric acid, ascorbic acid, aspartic acid, benzoic acid, potassium dihydrogen phosphate, sodium hydrogen phosphate, and the like.

In embodiments, the injectable compositions of the present invention do not require the presence of any additional preservative to remain sterile, or any agent (such as an antioxidant) to remain stable.

It has been surprisingly found by the present inventors that a self-preserved paricalcitol formulation can be attained by utilizing a vehicle comprising one or more of the vehicles as described herein. Further, such compositions possess excellent stability, both physical and chemical, and remain in their original state without significant precipitation of the active agent either during preparation or upon storage.

In an embodiment, the injectable compositions of the present invention comprise the vitamin D compound in a dissolved state, or a dispersed state such as an emulsion or a colloidal dispersion. When the vitamin D compound is in a dissolved state, one or more organic or aqueous solvents or oils is useful. When the vitamin D compound is in a dispersed state, the composition additionally comprises a surfactant.

The compositions of the present invention are ready-to-use or can be made into a lyophilized powder which can be reconstituted with suitable fluids known in the art, such as water for injection or normal saline, prior to administration. Alternatively, the compositions can be in the form of a micro-emulsion or an emulsion preconcentrate, which composition comprises at least one pharmaceutically acceptable oil or an oily substance, and which has an appreciable syringeability, and which forms a micro-emulsion or an emulsion upon contact with aqueous fluids.

In an embodiment, the present invention provides substantially alcohol-free, and/or substantially surfactant-free, and/or substantially preservative-free, injectable compositions comprising a therapeutically effective amount of a vitamin D compound such as paricalcitol, a vehicle comprising a glycol derivative or an organic solvent acceptable for parenteral administration, water, and optionally one or more pharmaceutically acceptable excipients.

It should, however, be understood that the excipients such as alcohols, surfactants or preservatives may be present in the compositions of the present invention in small parenterally acceptable amounts, such that the amounts do not cause any substantial associated toxicity, irritation or other side-effects in a subject to whom such composition is administered.

In a further embodiment, the present invention provides injectable compositions comprising vitamin D compounds in a single-use or multi-use container. When formulated as a multi-use composition, the formulation comprises the vitamin D compound such as paricalcitol in a suitable vehicle comprising one or more solvents, wherein the vehicle itself acts as a self-preserved system, or additionally comprises one or more preservatives, and optionally pharmaceutically acceptable excipients.

In yet another embodiment, the pharmaceutical injectable compositions of a vitamin D compound such as paricalcitol are stored in containers with a polymer stopper comprising a polymer comprising butyl, bromobutyl, ethylene-propylenediene monomers, or polyisoprene, or mixtures thereof, and wherein the container is a glass vial or a syringe. The pharmaceutical injectable composition of the present invention can be packaged into a flip-top vial, a pre-filled syringe, or a preloaded syringe.

In a further embodiment, the injectable compositions of the present invention may be provided in kits, wherein a kit comprises a lyophilized powder or a pre-concentrate comprising the vitamin D compound packaged, such as in a glass vial sealed with a stopper, and a suitable liquid for reconstitution packaged in a separate vial. Alternatively the composition may be provided as two separate components in a syringe, having one compartment containing a lyophilized powder or a pre-concentrate comprising the vitamin D compound, and having another compartment that contains a suitable liquid for reconstitution.

In yet another embodiment, the present invention provides processes for the preparation of injectable compositions comprising vitamin D compounds such as paricalcitol. In one embodiment, a process comprises mixing the vitamin D compound such as paricalcitol with the vehicle, and optionally adding one or more pharmaceutically acceptable excipients. In another embodiment, the pharmaceutical compositions according to the present invention provide compositions of vitamin D compounds such as paricalcitol that are aseptically filtered, such as through a filter membrane having 0.22 μm pores, filled into containers of desired capacities, and optionally terminally sterilized.

In a still further embodiment, the present invention provides methods of using the compositions of the present invention. Such methods comprise administration of an effective amount of an injectable composition according to the present invention to a subject in need thereof. The present invention also provides methods of prophylaxis or treatment of a kidney disease or disorder.

In a still further embodiment, the present invention provides uses of compositions prepared according to the present invention in the preparation of a medicament for the treatment of a kidney disease.

The pharmaceutical compositions according to the present invention comprising paricalcitol as the vitamin D compound may be used for the treatment secondary hyperparathyroidism in chronic kidney disease. “Effective amount” or “therapeutically effective amount” or “pharmaceutically effective amount” or “therapeutically useful amount” refer to the amount of a vitamin D compound such as paricalcitol that is effective to treat an intended disease or disorder such as secondary hyperparathyroidism in chronic kidney disease.

The following examples are provided solely for the purpose of further illustrating certain specific aspects and embodiments of the invention. Although embodiments of the invention have been disclosed for illustrative purposes, those skilled in the art will appreciate that various modifications, additions and substitutions are possible, without departing from the scope and spirit of the invention as herein described, and all are included within the scope of the invention.

### Example 1

### Paricalcitol Injection Composition

| Ingredient | Quantity/Unit Dose |
|---|---|
| Paricalcitol | 0.002 mg |
| Alcohol, dehydrated (ethanol) | 0.1 mL |
| Propylene glycol | 0.7 mL |
| Water for Injection | 0.2 mL |

Manufacturing Process:

1. Ethanol and water for injection were mixed well.

2. Paricalcitol was dissolved in propylene glycol.

3. Mixture of step 1 was combined with the mixture of step 2.

4. Mixture of step 3 was filtered through a 0.22 μm filter membrane and filled into vials.

### Example 2

### Paricalcitol Injection Composition

| Ingredient | Quantity/Unit Dose | |
|---|---|---|
| Paricalcitol | 0.004 | mg |
| Alcohol, dehydrated | 0.3 | mL |
| Propylene glycol | 1 | mL |
| Water for Injection | 0.7 | mL |

Manufacturing process is similar to that described in Example 1.

### Example 3

### Paricalcitol Injection Composition

| Ingredient | Quantity | |
|---|---|---|
| Paricalcitol | 0.05 | mg |
| Alcohol, dehydrated | 0.7 | mL |
| Propylene glycol | 4.3 | mL |
| Methyl paraben | 0.005 | mg |
| Propyl paraben | 0.002 | mg |

| | |
|---|---|
| Water for Injection | q.s. to 10 mL |

Manufacturing Process

1. Propyl paraben was dissolved in ethanol and methyl paraben was dissolved in water for injection, and the solutions were mixed together.

2. Paricalcitol was dissolved in propylene glycol.

3. The solution of step 2 was mixed with the solution of step 1.

4. Solution of step 3 was filtered through a 0.22 μm filter membrane and packaged into pre-filled syringes.

### Example 4

### Paricalcitol Injection Composition

| Ingredient | Quantity/Unit Dose | |
|---|---|---|
| Paricalcitol | 0.002 | mg |
| Propylene glycol | 0.5 | mL |
| Butylene glycol | 0.05 | mL |
| Water for Injection | 0.45 | mL |

Manufacturing Process:

1. Paricalcitol was dissolved in a mixture of propylene glycol and butylene glycol.

2. Mixture of step 1 was combined with water for injection.

3. Mixture of step 2 was filtered through a 0.22 μm filter membrane and filled into vials.

### Example 5

### Paricalcitol Injection Composition

| Ingredient | Quantity | |
|---|---|---|
| Paricalcitol | 0.025 | mg |
| Propylene glycol | 0.8 | mL |
| Polysorbate 80 | 0.01 | mg |

| | |
|---|---|
| Water for Injection | q.s. to 5 mL |

Manufacturing Process:

1. Polysorbate 80 was dissolved in water for injection.

2. Paricalcitol was dissolved in propylene glycol.

3. Mixture of step 1 was combined with the mixture of step 2.

4. Solution of step 3 was filtered through a 0.22 μm filter membrane and filled into vials.

### Example 6

### Paricalcitol Injection Composition

| Ingredient | Quantity | |
|---|---|---|
| Paricalcitol | 0.005 | mg |
| Alcohol, dehydrated | 0.35 | mL |
| Propylene glycol | 0.1 | mL |
| Water for Injection | 0.55 | mL |

Manufacturing process is similar to that described in Example 1.

### Example 7

### Calcitriol Injection Composition

| Ingredient | Quantity/Unit Dose | |
|---|---|---|
| Calcitriol | 0.005 | mg |
| Propylene glycol | 0.2 | mL |
| Sodium citrate | 0.001 | mg |
| Water for Injection | 0.8 | mL |

Manufacturing Process:

1. Calcitriol was dissolved in propylene glycol.

2. Sodium citrate was dissolved in water for injection.

3. Mixture of step 1 was combined with the mixture of step 2.

4. Solution of step 3 was filtered through a 0.22 μm filter membrane and filled into vials.

### Example 8

### Paricalcitol Injection Composition

| | Quantity/Unit Dose | | | |
|---|---|---|---|---|
| Ingredient | Option 1 | Option 2 | Option 3 | Option 4 |
| Paricalcitol | 0.005 mg | 0.005 mg | 0.005 mg | 0.005 mg |
| Propylene glycol | 0.2 mL | 0.3 mL | 0.4 mL | 0.5 mL |
| Water for Injection | 0.8 mL | 0.7 mL | 0.6 mL | 0.5 mL |

Manufacturing Process:

1. Paricalcitol was dissolved in propylene glycol.

2. Water for injection was combined with the mixture of step 1.

3. Solution of step 2 was filtered through a 0.22 μm filter membrane and filled into vials.

A physical evaluation of paricalcitol injection composition as described in Example 8 was conducted to ascertain the syringeability and stability of the composition. The results are presented in Table 1.

TABLE 1
Syringeability and stability data.

| Parameter | Example 8 (All options 1-4) |
|---|---|
| Syringeability* | 2 |
| Visual observation for precipitation | No precipitation (clear solution) |
| | observed for 24 hours |

*Syringeability was evaluated using a 21 gauge needle and based on the following scale: 1 = Good, 5-15 seconds; 2 = Fair, 20-30 seconds; and 3 = Poor, 60 seconds or more.

### Example 9

### Paricalcitol Injection Composition

| Ingredient | Quantity/Unit Dose | |
|---|---|---|
| Paricalcitol | 0.002 | mg |
| Alcohol, dehydrated | 0.07 | mL |
| Propylene glycol | 0.53 | mL |
| Water for Injection | 0.4 | mL |

Manufacturing Process:

1. Propylene glycol and alcohol were mixed.

2. Measured amount of water for injection was added to step 1.

3. Paricalcitol was added to step 2, and stirred.

4. Final volume was made up using water for injection.

5. Solution of step 4 was filtered through a 0.22 μm membrane filter and filled into vials.

We claim:

1. An injectable pharmaceutical composition comprising a vitamin D compound and at least one vehicle comprising more than about 50 percent by volume of a glycol, about 10 percent by volume or less of an alcohol, and about 40 percent by volume or less of water.

2. The injectable pharmaceutical composition of claim 1, wherein a vitamin D compound comprises paricalcitol.

3. The injectable pharmaceutical composition of claim 1, wherein a glycol comprises one or more of glycerin, polyethylene glycol, propylene glycol, and butylene glycol.

4. The injectable pharmaceutical composition of claim 1, wherein a glycol comprises propylene glycol.

5. The injectable pharmaceutical composition of claim 1, wherein an alcohol comprises a C₁ to C₈ aliphatic or aromatic alcohol.

6. The injectable pharmaceutical composition of claim 1, wherein an alcohol comprises one or more of ethanol, propanol, butanol, and benzyl alcohol.

7. The injectable pharmaceutical composition of claim 1, wherein a vehicle comprises about 53 percent by volume or more of glycerin, polyethylene glycol, propylene glycol, butylene glycol, or a mixture of any two or more thereof, about 7 percent by volume or less of ethanol, and about 40 percent by volume or less of water.

8. The injectable pharmaceutical composition of claim 1, comprising paricalcitol and a vehicle comprising about 53 percent by volume or more of a glycol, about 7 percent by volume or less of an alcohol, and about 40 percent by volume or less of water.

9. The injectable pharmaceutical composition of claim 1, packaged in a container having a stopper comprising one or more of a butyl polymer, a halogenated butyl polymer, a polymer from ethylene and propylenediene monomers, and polyisoprene.

10. The injectable pharmaceutical composition of claim 1, packaged in a container having a stopper comprising a bromobutyl polymer.

11. The injectable pharmaceutical composition of claim 1, packaged in a container comprising a vial or a pre-filled syringe.

12. A process for preparing an injectable pharmaceutical composition of claim 2, comprising: (a) dissolving a glycol in an alcohol; (b) adding water to the solution of (a); and (c) dissolving paricalcitol in the solution of (b).

13. A process for preparing an injectable pharmaceutical composition of claim 2, comprising: (a) dissolving paricalcitol in a glycol; and (b) adding water to the solution of (a).

14. An injectable pharmaceutical composition comprising paricalcitol and at least one vehicle comprising more than about 50 percent by volume of glycerin, polyethylene glycol, propylene glycol, butylene glycol, or a mixture of any two or more thereof, about 10 percent by volume or less of an alcohol, and about 40 percent by volume or less of water.

15. The injectable pharmaceutical composition of claim 14, wherein a vehicle comprises propylene glycol.

16. The injectable pharmaceutical composition of claim 14, wherein an alcohol comprises a Cᵢ to C₈ aliphatic or aromatic alcohol.

17. The injectable pharmaceutical composition of claim 14, wherein an alcohol comprises one or more of ethanol, propanol, butanol, and benzyl alcohol.

18. The injectable pharmaceutical composition of claim 14, wherein a vehicle comprises about 53 percent by volume or more of glycerin, polyethylene glycol, propylene glycol, butylene glycol, or a mixture of any two or more thereof, about 7 percent by volume or less of ethanol, and about 40 percent by volume or less of water.

19. A process for preparing an injectable pharmaceutical composition of claim 14, comprising: (a) dissolving glycerin, polyethylene glycol, propylene glycol, butylene glycol, or a mixture of any two or more thereof in an alcohol; (b) adding water to the solution of (a); and (c) dissolving paricalcitol in the solution of (b).

20. A process for preparing an injectable pharmaceutical composition of claim 14, comprising: (a) dissolving paricalcitol in glycerin, polyethylene glycol, propylene glycol, butylene glycol, or a mixture of any two or more thereof; and (b) adding water to the solution of (a).
